(19) United States
(12) Patent Application Publication
(10) Pub. No.: US 2015/0072315 A1
(43) Pub. Date: Mar. 12, 2015

(54) Formulations and kit for biometric deposition of apatite on teeth

(71) Applicant: Heraeus Kulzer GmbH, Hanau (DE)

(72) Inventors: Susanne BUSCH, Neu Anspach (DE); Andreas UTTERODT, Neu Anspach (DE); Michael GERLACH, Hofheim (DE)

(21) Appl. No.: 14/481,429

(22) Filed: Sep. 9, 2014

(30) Foreign Application Priority Data
Sep. 9, 2013 (DE) 10 2013 109 846.0

(51) Int. Cl. A61C 19/06; A61K 6/00; A61L 24/02

(52) U.S. Cl. CPC A61C 19/063; A61L 24/02; A61K 6/0017
USPC 433/217.1; 106/35

(57) ABSTRACT

The invention proposes formulations for biomimetic deposition of apatite from a partially elastic shaped body on teeth, whereby the shaped body a) contains at least one mineralization matrix containing a gel that comprises water-soluble phosphates or phosphates that can be hydrolyzed to form water-soluble phosphate ions and has a pH value of 2 to 8, optionally fluorides, and b) the at least one or a second mineralization matrix comprises a second gel having a pH value of 3.5 to 14 comprising calcium ions or compounds releasing calcium ions. Moreover, the method for producing the formulation for the deposition of apatite, in particular of needle-shaped fluorapatite crystals, is claimed. Using the formulations according to the invention, it is feasible to deposit more than or equal to 1 μm apatite, in particular fluorapatite, on tooth surfaces in order to seal or brighten porous tooth surfaces.

The invention proposes formulations for biomimetic deposition of apatite from a partially elastic shaped body on teeth, whereby the shaped body a) contains at least one mineralization matrix comprising a gel that comprises water-soluble phosphates or phosphates that can be hydrolyzed to form water-soluble phosphate ions and has a pH value of 2 to 8, optionally fluorides, and b) the at least one or a second mineralization matrix comprises a second gel having a pH value of 3.5 to 14 comprising calcium ions or compounds releasing calcium ions. Moreover, the method for producing said formulation is claimed. Using the formulations according to the invention, it is feasible to deposit equal to 1 μm or more apatite, in particular fluorapatite, on tooth surfaces in order to seal or brighten porous tooth surfaces.

Teeth are hard biomaterials in the form of composites based on proteins and apatite comprising calcium and phosphate. Enamel, i.e. the outer layer of the crown of the tooth, is the hardest part of the tooth and contains no viable cells. It consists of inorganic crystals which typically are present in highly oriented arrangements. Enamel is a tissue, which, once it is produced, remains nearly unchanged for life since the cells involved in building up the teeth die as soon as tooth formation is completed. Finished enamel consists of approx. 95% by weight apatite, approx. 3% by weight proteins and lipids, and approx. 2% by weight water.

In order to prevent or repair tooth damage, in particular due to caries, attempts to use remineralizing systems have been made for a long time. These initially involved the application of calcium phosphate compounds to improve the properties of the teeth.

Such one-component systems attempting to apply pre-made tooth substance, for example apatite, hydroxyapatite or other calcium phosphate compounds, to the teeth, are described, inter alia, in EP 0 666 730 B1 or WO 01/95863. It is a phe problem of said systems that treating the tooth substance with calcium phosphate compounds does not lead to the growth of an apatite that is structurally similar to the tooth substance, but rather to mere deposition of apatite crystals on the tooth substance, whereby the morphology of the apatite crystals is very different morphology from that of tooth substance. Accordingly, there is no strengthening effect on the enamel and no permanent filling of lesions, since the deposited apatite crystals do not comprise sufficient similarity and adhesion to the tooth substance.

Due to modern dietary habits, which often involve acidic food items, erosions of the dental hard substance that are not due to bacteria are on the rise [Dentale Erosionen: Von der Diagnose zur Therapie, Adrian Lussi, Thomas Jaeggi, Quitessenz Verlag].

But not only food items such as strongly acidified sweets, soft drinks or alcopops play a role in this regard, but the trend towards nutrition containing more fruit can also lead to dental problems. The continuous exposure to acids makes the enamel thinner and more porous. In extreme cases, the enamel can be dissolved totally and/or abraded such that the sensitive dentine is exposed. In the neck region of the teeth, in particular, which is protected by a very thin layer of enamel only, this occurs frequently. Acid-caused erosion can then proceed at even faster rates since dentine is more acid-soluble than enamel and wedge-shaped defects in the dental hard substance are often caused. Exposed dentine leads to sensitive, pain-sensitive teeth. However, sensitive dental necks can just as well be a consequence of inappropriate brushing habits. Increasing age is another reason for the enamel getting thinner. Habitual bruxism can also abrade the enamel at the incisal edges. Due to the improved prophylaxis in dentistry and strict addition of fluoride to most toothpastes and additional care products, caries is decreasing, but since the population in the industrialized countries is ageing and functional teeth have to work longer, the significance of non-cariogenic losses of dental hard substance is increasing as well.

Some forms of administration described to be suited for inducing the mineralization of apatite on the surface of teeth are known. U.S. Pat. No. 6,521,251 describes a composition that contains not only carbamide peroxide, but also calcium phosphates which are slightly more soluble than apatite, such as mono-, di- or tricalcium phosphate. But still, all these calcium phosphates are poorly water-soluble, such that the tooth cleaning means described are expected to have an abrasive rather than a remineralizing effect. In fact, U.S. Pat. No. 5,851,514 describes, inter alia, the addition of dicalcium phosphate as an abrasive.

U.S. Pat. No. 6,419,905 mentions the addition of potassium salts (e.g. citrate) and fluoride to the peroxide. Fluoride is suited for binding, in particular, calcium and phosphate ions from the saliva, leading to the precipitation of fluorapatite. If no other ions are added, the formation of CaF₂ has also been observed. Calcium fluoride particles can be stored in the plaque and can release fluoride for extended periods of time since they are more soluble than the apatite of the hard dental substance. However, conscientious repeated daily cleaning of the teeth largely removes the plaque to a large extent. Accordingly, the effect of calcium fluoride is short-lived and the fluoride-containing products need to be applied in regular intervals. No formation of new apatite has been observed with products of this type.

Patent JP20000051804 describes the concurrent use of concentrated phosphoric acid, conc. H₂O₂, and fluorapatite powder. The use of concentrated phosphoric acid in this context appears questionable as this substance can dissolve healthy enamel to a notable degree. Moreover, the bleaching solution is strongly irritating and must not contact the gingiva, although this is true, at a lesser level, of all tooth-bleaching agents having an oxidative effect. Moreover, repeated application does not lead to the build-up of a mineralization layer.

An acid-free application is described in U.S. 20050281759. Calcium peroxophosphate is proposed as essential ingredient in this context. The underlying rationale being that a single substance is to have the brightening and remineralizing effect, since the release of calcium and phosphate ions is triggered parallel to the oxidation. It is not clear whether or not the salts can attain any significant build-up of apatite during their relatively short period of action. U.S. Pat. No. 6,303,104 describes an oxidant-free two-component system consisting of soluble calcium and phosphate salts, which is claimed to have a brightening effect as well. The brightening is said to be caused through the addition of sodium gluconate, which forms complexes with staining metal ions (e.g. iron) from the enamel. Mixing of the components is expected to immediately lead to precipitation of the poorly-soluble calcium phosphates and it is not obvious why there should be pronounced remineralization, even more so as the product is just a toothpaste to which the tooth surfaces is exposed for no more than a few minutes at a time. U.S. Pat. No. 6,102,050 describes a dental floss having titanium dioxide particles that is said to have a brightening, remineralizing , and desensitising effect on the interdental surfaces. Titanium dioxide microparticles of a size of 0.1 to 1.5 μm are to act both as a mild abrasive and are to be absorbed by the enamel, which is said to be associated with a brightening effect. Presumably, the particles can no more than get incorporated mechanically into suitable hollow spaces which does not promise to lead to stable anchoring and can have no more than a temporary effect.

All patents described thus far fail to take into consideration that bio-minerals attain their high degree of structural organization and stability only because they are formed in the presence of specific biomolecules that define the formation of the micro- and macro-structure.

WO 2005/027863 describes a tooth care product that is said to possess a cleaning, remineralizing , desensitizing, and brightening effects. The nano-scale apatite-gelatine composite proposed as active component for remineralization and brightening precipitates in the presence of an aqueous gelatine solution and thus has polypeptides incorporated into it. This material is said to form a protective layer of dentine-like structure on the surface of the tooth due to so-called “neo-mineralization”, whereby the protective film is said to smoothen the surface and to be able to seal open dentine tubules. This effect is not comprehensible for a toothpaste, since said tooth care product preferably contains only 0.01-2% by weight “nanite” (WO 01/01930). The active substances can act for no more than a few minutes daily.

Any significant or surface-covering deposition of mineral is doubtful. Moreover, no deposition of mineral on enamel is described. No continuous increase in the thickness of the film upon extended application of the care product is described either. Moreover, the porous, poorly ordered structure of dentine is not capable of protecting the tooth from corrosive attacks. The commercially available product, Tooth Mousse or Mi-Paste, is based on patent specifications by Reynolds [WO 98/40406] and is said to remineralize porous enamel. The invention is based on casein (CPP) having a stabilizing effect on amorphous calcium phosphate (ACP). In contact with the hard dental substance, the CPP-ACP agent is to remineralize into hydroxylapatite. A protective film of dentine-like structure of this type appears unsuited to provide long-term protection.

It is common to all patents that they only refer to remineralization without documenting same and/or without having a desensitizing effect. US 2012/0027829A1 describes the formation of hydroxylapatite layers (HAP) on dentine by repeatedly applying pasty mixtures of propylene glycol, glycerol, xylitol, polyethylene glycol, cetylpyridiniumchloride, tetracalcium phosphate, and an alkali salt of phosphoric acid to the teeth. Since tetracalcium phosphate reacts immediately with phosphoric acid salts in the presence of water, two separate pastes are produced first and mixed only right before application. No formation of HAP on enamel is described and no data is provided on the layer formed, which also was not reproducible in own experiments.

The technique described in US2005220724 and DE 10 2004 054 584.7 provides a fluorapatite layer which possesses enamel-like strength and increases in thickness upon repeated application. Water-soluble phosphate and fluoride salts are incorporated in the buffered gel A, whereas calcium ions are incorporated in gel B. Optionally separated through an ion-free protective layer, the gelatine gels, which are solid at physiological temperature, are applied, while heating, to the tooth surfaces one after the other. An increase of the thickness of the layer as a function of the exchange cycles of the gels can be observed. The growth rates are 0.5 to 5.0 μm/day. The biological structures of the tooth substance are replicated individually by the fluorapatite; hollow spaces due to open dentine tubuli are sealed after a few exchange cycles.

Regarding the use in humans, it is inconvenient that the gels need to be heated before application. The application of the second and third gel layer may cause underlying, previously applied gel layers to liquefy again and mix with the upper layers in undesirable manner. Small amounts applied as described, in particular, dry out quickly upon exposure to air and are then difficult to liquefy by heating them. The method hardly allows exactly defined amounts of gel of even thickness to be applied to the tooth. Moreover, the three gel layers, each being up to 6 mm in thickness, are quite bulky, which leads to problems in the case of protective systems, such as splints or plasters, as space for large gel reservoirs needs to be created in this case.

The method disclosed above becomes increasingly laborious when the entire jaw including all tooth surfaces is to be treated. Since an application period for the formation of fluorapatite should not be less than 8 hours under ideal conditions, it would be of advantage if the patient could use the system himself/herself by using it before going to bed. For this, the patient would have to warm up the gels and place them precisely on the teeth, which is very difficult since warmed-up liquid gelatine is very tacky. Moreover, this is associated with a major risk of burns. It is also disadvantageous that the gels stay liquid and do not safely adhere on the teeth in the oral environment.

Since the gels leak despite the presence of protection, such as, e.g., an individualized deep-drawing splint, the splint needs to be sealed with a suitable sealing system, which renders the method even more complicated.

The use of pre-made gel strips according to DE 10 2006 055 223 A1 is advantageous in that there is no cumbersome heating involved and the strips are of the same thickness. However, one major disadvantage is that the strips reach only partial regions of the teeth. However, since erosions can basically affect all surfaces of teeth, it would be desirable to have the mineralization kit exert its effect in all places. Moreover, it is very cumbersome to unpack the two strips and to insert them, for example, into a deep-drawing splint, which, in addition, also needs to have a reservoir for the gel strips. The issue of sealing the splints is not solved satisfactorily. Since the strips liquefy in the oral environment, sealing is required though in order to prevent the agents from leaking into the oral space.

The invention describes a method based on patents US2012195941(A1) and US2008241797 (A1) that can be used to apply an enamel-like fluorapatite layer onto teeth. In the embodiment described, two mineralization strips are formed appropriately such that individual tooth surfaces, a group of teeth or multiple tooth surfaces can be treated at once. It is also feasible to treat tooth surfaces of entire jaws.

The invention is based on the object to be able to provide formulations for a mineralization kit that produces high-quality enamel-like coatings made of apatite on hard dental substance at high reproducibility aiming to protect the hard dental substance from excessive loss of enamel. The ingredients are to be much like the biological original and the application should be as simple as possible. Further objects were to simplify the application significantly, preferably, the application should be possible either at the dentist's and directly by the user. Moreover, the application time of a single use of a kit was to be increased. The object was therefore to develop formulations that comprise mostly biomolecules or compounds of biological origin, are particularly well-tolerated for prolonged application, and do not lead to gingival irritation. Moreover, the time of an advantageously surface-covering deposition was to be reduced, i.e. the growth rate of the apatite was to be affected beneficially. Moreover, the issue of sealing of the gels should preferably be solved and a formulation should be provided, which preferably needs no separate sealing of the dental splint without preventing the deposition of the apatite.

The objects are solved through a formulation according to claim 1 and a kit according to claim 19 as well as through the methods for producing the formulation according to claims 14 and 15.

The objects were solved in that the at least one composite mineralization matrix based on denatured collagen or other gel-forming agents and mineral substances is produced with a 2-hydroxycarboxylic acid having a hydrocarbon residue. In this context, 2-hydroxycarboxylic acids possessing good solubility in water and, in particular, in the gel-forming agents, are preferred. Preferably, the acid has a pKs value of 3 to 6. Preferably, Tris buffer is used in the calcium ions-containing gel and the pH value is adjusted to a range of 4 to 14, in particular 6 to 11, particularly preferably to 7.2 to 9.0. Advantageously, the phosphate ions-comprising gel is adjusted to a pH value in the range of 2 to 8, more preferably 2 to 5.5 or 3.7 to 5.7, using a sodium acetate buffer.

It has been evident that the apatite layers grow better when formulations containing lactic acids rather than other carboxylic acids are used. In application tests, which also included formulations produced with acetic acid, it has been evident that the lactic acid formulations were tolerated better. This is related, in part, to formulations produced with acetic acid or other odorous acids are perceived as being more unpleasant.

The tolerability of the carboxylic acids is essential to the application, since the formulations remain in the mouth for several hours and should not lead to any skin or mucosal irritations. Another advantage of having lactic acid in the formulations according to the invention is its preservative effect. In order for weak acids to have a preservative effect, the pH of the formulation must be acidic, since only non-dissociated molecules can penetrate through the cell membrane into the inside of microorganisms.

It is therefore particularly preferred according to the invention to use lactic acid in the phosphate component.

According to an alternative, it is preferred to make the mineralization matrix insoluble in the oral environment by means of chemical modification, in particular as a formulation with separate shaped bodies or as a multi-part shaped body each comprising at least one or two mineralization matrices. Surprisingly, the mineralization activity is affected beneficially despite the modification, since the gel no longer spreads in the oral environment and can act evenly during the entire duration of treatment. Said chemical modification preferably proceeds by means of at least partial chemical cross-linking at the surface of the mineralization matrix such that at least one partially chemically cross-linked plane or partial envelope (casing) is formed. According to the invention, the cross-linked planes or the envelope act like a membrane through which aqueous media, such as saliva, can penetrate into the mineralization matrix and, concurrently, apatite can be deposited on the tooth surfaces outside of the mineralization matrix.

According to the invention, the plane or envelope formed can, on the one hand, adapt optimally to the surfaces of the teeth when the material swells in the mouth due to it being a thin layer and, on the other hand, also favours the deposition of apatite at the approximal spaces. It is another particular advantage of the superficial cross-linking of the mineralization matrix, during which a shaped body is formed, that an at least partially elastic shaped body is formed which can adapt optimally to the surface contour of teeth and rows of teeth. The partially elastic shaped body of the mineralization matrix adapts to the tooth surfaces particularly well as it is more easily deformed at body temperature in the oral environment. In order to affix the shaped body, comprising at least one mineralization matrix, optimally to the buccal, labial, mesial and/or approximal surfaces of the teeth, it is advantageous to place and/or to provide the at least one shaped body in a dental splint. Since the shaped body according to the invention is affixed with a dental splint, apatite can be deposited, at least on part or almost completely, on the teeth of the upper and lower jaw in buccal, mesial, labial, palatinal, distal and approximal position. The use, according to the invention, of the at least one shaped body allows, easily and for the first time, for biomimetic remineralization of at least, partially, equal to 1 μm or more, preferably of more than or equal to 2 μm, preferably of more than or equal to 3 μm through inserting the shaped bodies into a splint and placing the splint onto the teeth over night, for example for approx. 8 to 12 hours, optionally up to 16 or 24 hours, alternatively during the day as well, whereby biomimetic remineralization of at least partially, preferably on average, from 1 to 10 μm, 1 to 5 μm is particularly preferred. It is particularly preferred for the remineralization to occur in two-dimensional manner in an area and with the thickness of the layer formed being as homogeneous as possible.

Surprisingly, it has been found that the mineralization product forms more evenly and more densely on the tooth surface after the chemical stabilisation. The cross-linked plane or envelope is so porous that comparable apatite layers can be deposited despite the formation of the envelope, as is shown in examples 2 to 4 and 6 according to the invention by comparison to examples 1 and 5. Presumably, the chemical modification leads to the formation of a less temperature-sensitive and preferably less hydrolysis-sensitive network of polypeptides around the mineralization matrix, whose pores are large enough, though, to still allow molecules and ions forming the stable, ordered apatite layer on the tooth surface to pass. The chemically cross-linked layer or plane basically acts like an ion- and molecule-permeable membrane.

A sufficiently thick, homogeneous, and stable apatite layer can be attained by the optimal interplay of the components: mineral salts, buffer, and pH value. The pH value can be adjusted in particularly tolerable manner for the user through the use of lactic acid according to the invention. It is crucial to select the concentrations correctly at a corresponding degree of cross-linking. The formulation according to the invention can be used for depositing apatite on teeth or any other biological surfaces, such as a bone matrix.

Since the application of the formulations according to the invention is simpler, the system can be used both by dentists on patients and by the patients on themselves. The deposition of a fluoride-rich calcium phosphate layer can reduce sensitivities on the teeth, it can reduce cracks and initial porosities and increases the acid stability of the teeth. Initial losses of hard dental substance due to acid erosion can be stopped and/or partially or completely reversed. The increased fluoride content in the layers as compared to untreated teeth reduces the solubility of the newly formed mineral. The natural enamel is protected from toothbrush erosion by the protective layer.

The object of the invention is a formulation for the deposition of apatite, in particular well-suited for biomimetic deposition of apatite, selected from fluoroapatite (Ca₅[F](PO₄)₃), hydroxylapatite (Ca₅[F](PO₄)₃) or mixtures thereof on teeth or on a bone matrix of vertebrates, whereby the formulation comprises at least one partially elastic shaped body, in particular three-dimensional, preferably flat shaped body, e.g. board shaped body, comprising at least one mineralization matrix containing at least one gel, whereby the gel comprises at least one carboxylic acid or a mixture of carboxylic acids selected from 2-hydroxycarboxylic acids substituted on C-2 by a hydrocarbon, and

a) the at least one mineralization matrix comprises a gel containing water-soluble phosphates or phosphates that can be hydrolyzed to form water-soluble phosphate ions and has a pH value of 2 to 8 (phosphate component), and

b) the at least one or a second mineralization matrix comprises a second gel comprising calcium ions, in particular compounds containing water-soluble calcium ions, or compounds releasing calcium ions (calcium component) having a pH value of 3.5 to 14, comprising, comprising calcium ions or compounds releasing calcium ions (calcium component),

particularly preferably the at least one mineralization matrix is present in a first shaped body I. and the second mineralization matrix is present in a second shaped body II., alternatively two mineralization matrices are present in one shaped body.

Preferably, the shaped body comprises a reduced solubility in aqueous media as compared to the mineralization matrix in at least one plane, at least partly.

Carboxylic acids selected from 2-hydroxycarboxylic acids substituted on C-2 by a hydrocarbon residue preferably comprise, as hydrocarbon residue, substituted or non-substituted hydrocarbon residues, preferably linear, branched and/or cyclical alkyl, alkylaryl, alkylene, alkylenearyl or aryl groups having 1 to 10 C atoms, whereby non-substituted alkyl and/or aryl groups are particularly preferred. Due to the better solubility, short-chain alkyl groups are particularly preferred as hydrocarbon residue on C-2. Alkyl groups having 1 to 10 C-atoms, preferably having 1, 2, 3, 4, 5 or 6 C atoms are particularly preferred.

Particularly preferred carboxylic acids according to the invention comprise a pKs value of 3 to 6, in particular of 4 to 5. Specifically preferred carboxylic acids are selected from (S)-2-hydroxycarboxylic acids, (R)-2-hydroxycarboxylic acids, (RS)-2-hydroxycarboxylic acids or mixtures containing these, particularly preferred is the 2-hydroxycarboxylic acid selected from 2-hydroxypropanoic acid, (R-)-lactic acid, (S)-lactic acid, (RS)-lactic acid, (S)-2-hydroxy-3-methyl-butandioic acid, (R)-2-hydroxy-3-methyl-butandioic acid, (RS)-2-hydroxy-3-methyl-butandioic acid, (S)-2-hydroxy-pentanoic acid, (R)-2-hydroxy-pentanoic acid, (RS)-2-hydroxy-pentanoic acid, (S)-2-hydroxy-pentandioic acid, (R)-2-hydroxy-pentandioic acid, (RS)-2-hydroxy-pentandioic acid, (D)-2-phenyl-2-hydroxyethanoic acid, (L)-2-phenyl-2-hydroxyethanoic acid, (DL)-2-phenyl-2-hydroxyethanoic acid (D- and L-mandelic acid) or mixtures containing these and mixtures containing at least two of the aforementioned acids or phosphate buffer. Appropriately, 2-hydroxy-3-methyl-butandioic acid (citramalic acid), 2-hydroxy-pentanoic acid, 2-hydroxy-pentandioic acid can be used.

According to a preferred embodiment of the invention, the at least one mineralization matrix or the two mineralization matrices each independently comprising at least one gel are present in the form of a flat element (e.g. two-dimensional element, element of an area) or of an at least partial negative image of the jaw. It is also preferred that the at least one or two partially elastic shaped body/bodies is/are also present in the form of a flat element or of an at least partial negative image of the jaw.

It is preferred that the aforementioned at least one plane is a plane arranged on the outer surface or is an essentially complete outer envelope.

Formulations according to the invention comprise one or two shaped bodies in the form of a flat element each having one enclosing envelope having reduced solubility that functions, in particular, as a membrane. In an alternative embodiment, the formulation comprises a shaped body in the form of a flat element each having an enclosing envelope having reduced solubility that functions, in particular, as a membrane, having two mineralization matrices situated one on top of the other each comprising one gel, whereby one gel comprises the phosphate component and one gel comprises the calcium component. Said two mineralization matrices are comprised by the shaped bodies A and B of the kit. According to another alternative embodiment, a flat element comprises a mineralization matrix having a gel, whereby the gel optionally is sub-divided by a membrane layer into two reservoirs, one comprising the phosphate component and one comprising the calcium component. The aforementioned shaped bodies are preferably present as flat element with a layer thickness of 5 to 6,000 μm, whereby mineralization matrices in the form of a flat element each having a layer thickness of 5 to 3,000 μm are particularly preferred, preferably 100 to 600 μm, in particular 300 to 600 μm or about 500 μm plus/minus 200 μm, in particular plus/minus 200 μm, advantageously plus/minus 100 μm or better. The shaped bodies according to the invention comprise, at least partially, an envelope or at least one plane of reduced solubility. Instead of a flat element, the shaped body can just as well be present in the form of at least a partial negative image of the jaw, preferably as a negative mould of the teeth of the upper and/or lower jaw. Flat elements of the calcium component are preferably present having a layer thickness of 500 to 1,500 μm, particularly preferable are mineralization matrices in the form of a flat element each having a layer thickness of 500 to 1,200 μm or about 1,000 μm plus/minus 100 μm, in particular plus/minus 50 μm. Flat elements of the phosphate component are preferably present having a layer thickness of 100 to 1,000 μm, particularly preferable are mineralization matrices in the form of a flat element each having a layer thickness of 150 to 800 μm, preferably 300 to 800 μm, in particular 400 to 600 μm or about 500 μm plus/minus 100 μm, in particular plus/minus 50 μm.

Formulations according to the invention have a water content after cross-linking, and optionally after subsequent drying, of 8 to 60% by weight, preferably of 30 to 55% by weight. It is also preferred for the gels of the formulations according to the invention to have a water content after cross-linking, and optionally after subsequent drying, in the mineralization matrix of the phosphate component of 20 to 40% by weight, preferably of 25 to 35% by weight, particularly preferably about 30% by weight with a deviation of plus/minus 5% by weight. Accordingly, formulations of the calcium component that have a water content after cross-linking, and optionally after subsequent drying, in the mineralization matrix of 30 to 60% by weight, preferably of 40 to 60% by weight, more preferably about 50% by weight with a deviation of plus/minus 5% by weight are preferred. The formulations thus produced can subsequently be welded into blisters, sachets or the like such as to be air- and moisture-tight.

Preferably, the following alternatives of the shaped bodies can be present: a) two shaped bodies, in particular each having a partial, preferably essentially complete, envelope, particularly preferably having an enclosing envelope, preferably two shaped bodies with a cross-linking of the upper and/or lower side of the shaped bodies that are present as flat element, b) a shaped body having two mineralization matrices, preferably separated through a membrane, c) a shaped body having a mineralization matrix comprising two gel regions that are optionally separated through a membrane. Using two mineralization matrices or two gel regions for the phosphate component allows a concentration gradient to be adjusted in the shaped body. Accordingly, a concentration profile can also be adjusted for a shaped body containing the calcium component. The envelope or the plane possesses reduced solubility in aqueous media as compared to the mineralization matrix, preferably the envelope functions as a membrane and reduces the dissolution of the mineralization matrix in the oral environment. However, H₂O from saliva can penetrate and fluorides, calcium, and phosphate ions as well as composites or polypeptides can diffuse through the envelope and can be deposited on the tooth surface as apatite, hydroxylapatite or fluorapatite. The envelope encloses a water-rich gelatine matrix from which the composites, ion-loaded water molecules and/or hydrated ions can diffuse. The diffusion of the composites is important since the composites are organic macromolecule-substituted hydroxyapatites that form the enamel, and in order to deposit these on the tooth surfaces. According to the invention, fluorapatite-protein composites from the shaped bodies are deposited on the tooth surfaces.

The fluorapatite deposited from the formulation according to the invention is preferably present on the teeth in crystalline form, preferably micro-crystalline, particularly preferably in the form of needle-shaped crystallites. The at least partial apatite layer, preferably contiguous apatite layer, deposited on the tooth surfaces in the course of one application cycle of approx. 8 hours has a layer thickness of at least 1 μm. The scope of the invention also includes at least partially non-contiguous apatite layers that cover at least a part of the treated tooth surface irregularly to preferably homogeneously. The apatite layers can, at least partially, be up to 15 μm, preferably essentially homogeneous, which is preferred, apatite layers of more than or equal to 1 μm or more, more preferably more than or equal to 2 μm, even more preferably more than or equal to 5 μm to 13 μm, and on average of 1 to 10 μm are obtained.

Reduced solubility of the chemically cross-linked plane or envelope with respect to aqueous media as compared to the mineralization matrix shall be understood as follows: the mineralization matrix not chemically cross-linked and, optionally, the mineralization matrix modified with a plasticizer only, for example the gelatine cross-linked to glycerol as adduct via hydrogen bonds. Preferably, the following gelatine qualities are used: Bloom 175 to 300 or higher with Gelatine Bloom 300 (pork rind) being preferred.

Teeth of vertebrates include human teeth, prostheses of human teeth, deciduous teeth (Dentes decidui), permanent teeth (Dentes permanentes), crowns, inlays, implants, teeth of animals, such as domestic and livestock animals, such as dogs, horses, cats.

In the scope of the invention, an at least partially elastic shaped body shall be understood to mean a three-dimensional shaped body that is present as flat element or at any three-dimensional geometry, in particular in the form of an at least partial negative image of the jaw, and has elastic properties. The shaped body shall be considered to be elastic or partly elastic if the body changes its shape when exposed to a force and returns to its original shape, partly or fully, when the force ceases to act on it. The shaped body preferably possesses the property of being elastic or partly elastic when it is applied in the oral environment and preferably after production. The elasticity may decrease upon excessive drying.

In a formulation that is particularly preferred according to the invention, the mineralization matrix comprises in a) the following composition: a) the at least one mineralization matrix comprises a gel comprising (i) water-soluble phosphates or phosphates that can be hydrolyzed to form water-soluble phosphate ions, in particular Na₂HPO₄, whereby the phosphate content in the mineralization matrix preferably is 1 to 10% by weight, more preferably 2 to 8% by weight, particularly preferably 5 to 8% by weight, (ii) a content of water or of a mixture of water and an organic solvent, (iii) optionally at least one 2-hydroxycarboxylic acid having a hydrocarbon residue on C-2, in particular a 2-hydroxycarboxylic acid having an alkyl residue with 1, 2, 3, 4 or 5 C-atoms on C-2, according to the invention this would be lactic acid, and optionally a buffer system, whereby, in particular, a buffer system is present for adjusting the pH value in the range of 2 to 8, in particular of 3.5 to 8, preferably of 3.5 to 6, particularly preferably 5.5 plus/minus 0.5. The content refers to PO₄³⁻.

Concurrently, the particularly preferred formulation according to the invention comprises in b) a mineralization matrix of the following composition: the second mineralization matrix or the at least one mineralization matrix comprises a second gel comprising (i) calcium ions or compounds releasing calcium ions, in particular calcium dichloride or hydrates thereof, preferably in addition calcium sulfate, nanoapatite, sodium carbonate or calcium oxalate, whereby the calcium content in the mineralization matrix preferably is 1 to 10% by weight, more preferably more than or equal to 1.5 to 7.5% by weight, (ii) optionally water or a mixture of water and an organic solvent, and (iii) optionally 2-hydroxycarboxylic acid having a hydrocarbon residue on C-2, in particular a 2-hydroxycarboxylic acid having an alkyl residue having 1 to 10 C-atoms, preferably 1, 2, 3, 4 or 5 C-atoms, on C-2, according to the invention this would be lactic acid, and/or a buffer system.

It is preferred to use hydroxycarboxylic acids, such as fruit acids and alkali salts, to produce the buffers. The content refers to calcium (Ca²⁺).

Moreover, it is preferred that the formulation comprises a gel in the at least one mineralization matrix, whereby the gel comprises at least one water-soluble fluoride (F⁻), with fluoride ions or a compound releasing fluorides. Particularly preferably, the formulation in a) comprises as further component (iv) at least one water-soluble fluoride or one compound releasing fluorides.

According to a preferred refinement of the invention, the at least one water-soluble fluoride or the at least one compound releasing fluorides comprises, in particular in a), the at least one mineralization matrix, (i) at least one non-substituted or substituted alkyl groups-comprising quaternary mono- or poly-ammonium compound, preferably having four substituted alkyl groups, whereby the at least one substituted alkyl group comprises hydroxyalkyl, carboxyalkyl, aminoalkyl groups having 1 to 25 C-atoms or organo-functional, hetero atom-interrupted groups having up to 50 C-atoms. Preferred ammonium compounds can contain 1 to 20 quaternary ammonium functions, preferably 1, 2, 3, 4, 5, 6, 7, 8 ammonium functions; it is preferable to use Olaflur (N,N,N′-tris(2-hydroxyethyl)-N′-octadecyl-1,3-diaminopropandihydrofluoride) as water-soluble fluoride. Also preferred are aminefluorides, such as Oleaflur (C₂₂H₄₅FNO₂), Decaflur (9-Octadecenylaminhydrofluoride), ethanolamine hydrofluoride, (ii) a fluorides-releasing organo-functional amino compound or a fluorides-releasing antiseptic based on organo-functional amino compounds, such as, in particular, fluorides of N-octyl-1-[10-(4-octyliminopyridin-1-yl)decyl]pyridin-4-imine, cetylpyridinium fluoride, or c) water-soluble inorganic fluorides, such as alkali fluorides, sodium fluoride, potassium fluoride, tin fluoride, ammonium fluoride, or fluorides-releasing inorganic fluorides, such as zinc fluoride, zinc hydroxyfluoride.

Preferably, at least one gel-forming agent selected from denatured collagen, hydrocolloids, polypeptides, protein hydrolysis products, synthetic polyamino acids, polysaccharides, polyacrylates (Superabsorber) or mixtures comprising at least two of the aforementioned gel-forming agents can be present in the formulation as gel according to the invention.

In a formulation according to the invention, the mineralization matrix comprises, as gel, gelatine and preferably a plasticizer, preferably a polyol, such as glycerol and/or conversion products thereof, optionally in the presence of water. Alternatively, gelatine and a plasticizer such as sorbitol can be used just as well. The effect of the plasticizer is to increase the melting range by forming intermolecular hydrogen bonds. According to the invention, gelatine (denatured collagen, animal protein, protein) is preferably used as gel and acid-hydrolyzed collagen is used particularly preferably or gelatine and a polyol such as glycerol.

For use on teeth, it is preferred that the mineralization matrix and the shaped body are present as a flat element or at least partial negative image of a jaw. A flat element according to the invention or the shaped body in the form of at least a partial negative image of the jaw can just as well reproduce a surface texture simulating the tooth surfaces of a dental arch.

Compounds for thermal stabilization of the gel preferably comprise plasticizers based on polyols.

The di- or poly-functional cross-linkers, preferably glutardialdehyde, are used as compounds for chemical cross-linking, in particular covalent cross-clinking, in the at least one plane or for forming the envelope of the mineralization matrix. The chemical cross-linkers form covalent cross-linking sites with the gels, in particular with the polypeptides or polyamino acids. Preferably, the di- or poly-functional cross-linkers comprise dialdehydes, polyepoxides and/or polyisocyanates as well as mixtures comprising at least two cross-linkers. Furthermore, it is preferred to use pharmacologically tolerable cross-linkers. Preferred dialdehydes comprise alpha, omega dialdehydes of hydrocarbons, in particular comprising 2 to 50 C-atoms, in particular 4 to 10 C-atoms in the di-functional alkylene group. Treating the mineralization matrix with a cross-linker reduces the solubility of the gelatine to a level such that it does not liquefy in the oral environment for approximately 8 hours.

Preferably, the treatment with glutardialdehyde proceeds for at least 5 s, depending on the application on hand the cross-linking may proceed for longer and thus be more pronounced, for example if a mineralization matrix is to remain in the oral environment for 12 to 16 hours. After rinsing for 40 s, immersion in or spraying of an 0.5% glutardialdehyde solution, the solubility of the gel is reduced to a level such that it does not liquefy in the oral environment for up to 8 hours. The membrane (layers) that can be used optionally are free of ions.

The cross-linker solution preferably has a cross-linker content of approx. 0.25 to 0.5% by weight, preferably of glutardialdehyde. It has been evident that the best results in terms of sufficient cross-linking and optimal permeability for the apatite composites to be deposited are obtained with a treatment time of 0 to 60 s, preferably approx. 5 to 40 s, particularly preferably 10 to 30 s, according to the invention about 20 seconds (s).

According to a preferred embodiment, the formulation comprises

(I) a shaped body in the form of a flat element having at least two mineralization matrices that are optionally separated by a membrane (layer), each in the form of a flat element containing the gel with the following layer structure in the shaped body:

A) a first mineralization matrix in the form of a flat element comprising gel water soluble and phosphates or phosphates that can be hydrolyzed to form phosphate ions, water-soluble fluorides or compound releasing fluorides, water or a mixture of water and an organic solvent, optionally at least one 2-hydroxycarboxylic acid having a hydrocarbon residue on C-2, in particular a 2-hydroxycarboxylic acid having an alkyl residue having 1 to 10 C-atoms, preferably having 1, 2, 3, 4, or 5 C-atoms on C-2, this would be lactic acid according to the invention, and optionally a buffer system;

B) optionally membrane (layer);

C) a second mineralization matrix in the form of a flat element comprising gel and calcium ions or compounds releasing calcium ions, optionally water or a mixture of water and an organic solvent, optionally at least one carboxylic acid and/or a buffer system.

Also a subject matter of the invention are formulations comprising (I) a shaped body in the form of a flat element having at least two mineralization matrices, optionally separated through a membrane, each independently in the form of a flat element containing the gel at a layer thickness of 50 to 6,000 μm, in particular of 500 to 2,000 μm, preferably of 1,000 to 2,000 μm or 500 to 1,500 μm, or (II) two separate shaped bodies each independently in the form of a flat element each having at least one mineralization matrix in the form of a flat element containing the gel, whereby each shaped body independently has a layer thickness of 10 to 3,000 μm, preferably of 50 to 1,000 μm, particularly preferably of 100 to 750 μm, even more preferably of 300 to 600 μm, yet more preferably of about 500 μm plus/minus 300 μm, whereby the first shaped body comprising phosphates, such as hydrogen phosphates, or phosphates that can be hydrolyzed to form water-soluble phosphates, has a layer thickness of 50 to 3,000 μm, in particular of 100 to 3,000 μm, preferably of 300 to 1,000 μm, particularly preferably of about 500 μm plus/minus 200 μm or +/−50 μm, and/or the second shaped body comprising calcium ions or compounds releasing calcium ions has a layer thickness of 10 to 3,000 μm, in particular 100 to 3,000 μm, preferably of 300 to 1,000 μm, more preferably of 300 to 750 μm, even more preferably of about 500 μm plus/minus 200 μm or +/−50 μm. Alternatively, the aforementioned shaped bodies are part of an at least partial negative image of a jaw with a corresponding multi-layered internal design that is to be arranged on the teeth.

In addition to lactic acid and the calcium salt of lactic acid, glycolic acid, aspartic acid, and malic acid can be used in mixtures also containing lactic acid or in mixtures also containing a calcium salt of lactic acid.

For the buffer systems, it is advantageous to use alkali and/or alkaline earth salts or zinc salts of the following carboxylic acids selected from fruit acids, such as α-hydroxycarboxylic acids such as malic acid, citric acid, glycolic acid, lactic acid, and tartaric acid; amino acids, fatty acids, hydroxycarboxylic acids, dicarboxylic acids, and mixtures comprising at least two of the aforementioned acids and/or the buffer system comprises carboxylates of alkylcarboxylic acids, fatty acids, fruit acids, fumarates, amino acids, hydroxycarboxylic acids, dicarboxylic acids, and mixtures comprising at least two of the aforementioned acids or phosphate buffer. It is advantageous to use alkali and/or alkaline earth salts or zinc salts for the buffer systems.

The buffer systems comprise EDTA, TRIS: tris(hydroxymethyl)-aminomethane for pH 7.2 to 9.0, HEPES: 4-(2-hydroxyethyl)-1-piperazinethanesulfonic acid for pH 6.8 to 8.2, HEPPS: 4-(2-hydroxyethyl)-piperazin-1-propansulfonic acid for pH 7.3 to 8.7, barbital-acetate buffer, MES: 2-(N-morpholino)ethansulfonic acid for pH 5.2 to 6.7, carbonic acid-bicarbonate system for pH 6.2 to 8.6; neutral, carbonic acid-silicate buffer for pH 5.0 to 6.2; weakly acidic, acetic acid-acetate buffer for pH 3.7 to 5.7, phosphate buffer: NaH₂PO₄+Na₂HPO₄ for pH 5.4 to 8.0, ammonia buffer NH₃+H₂O+NH₄Cl for pH 8.2 to 10.2, citric acid or citrate buffer. Particularly preferred buffer systems comprising lactic acid buffer systems, EDTA, or barbital-acetate buffer and, in the mouthwash, TRIS (tris(hydroxymethyl)-aminomethane) buffer. TRIS (Tris(hydroxymethyl)-aminomethane) is used in the mouthwash, which is a synonymous term for pre-treatment solution.

Phosphates that can be used according to the invention to produce the phosphate-containing mineralization matrices comprise phosphates, hydrogenphosphates or phosphates that can be hydrolyzed to form water-soluble phosphate ions, comprising

a) alkali phosphates, alkaline earth phosphates, dihydrogenphosphates, sodium dihydrogenphosphate, NaH₂PO₄, potassium dihydrogenphosphate, KH₂PO₄, hydrogenphosphates, dipotassium hydrogenphosphate, K₂HPO, disodium hydrogenphosphate, Na₂HPO₄, phosphate esters, monoesters, diesters, and triesters of phosphates, sodium phosphate, Na₃PO₄, potassium phosphate, K₃PO₄, calcium dihydrogenphosphate, Ca(H₂PO₄)₂, monoesters, diesters, and triesters of calcium hydrogenphosphate, CaHPO₄, calcium phosphate, Ca₃(PO₄)₂, and/or

b) the calcium ions or compounds releasing calcium ions comprise calcium chloride, calcium dichloride dihydrate, calcium salt of a carboxylic acid comprising alkylcarboxylic acids, hydroxycarboxylic acid, dicarboxylic acids, fruit acids, amino acids, such as calcium lactate, calcium gluconate, calcium lacto-gluconate, calcium-alginate, calcium-L-ascorbate, compounds releasing poorly water-soluble calcium ions in delayed manner comprising calcium sulfate, calcium apatite, calcium-carbonate, calcium oxalate, calcium phosphate, calcium alginate, preferably having a particle size of less than 100 μm, preferably about 10 μm, particularly preferably of less than or equal to 5 μm, for example up to 1 μm or 50 nm or preferably mixtures of water-soluble and poorly water-soluble calcium ions or compounds releasing calcium ions. The poorly water-soluble compounds releasing calcium ions in delayed manner are added to the gel containing calcium ions that are easily soluble in water in order to improve the texture of the, in some cases, tacky gels. A total of 1 to 50% by weight compounds releasing poorly water-soluble calcium ions, preferably 5 to 30% by weight with respect to the total composition of the mineralization matrix, can be used.

Also a subject matter of the invention are a method for producing a phosphate ion-containing formulation and a formulation obtainable according to said method for biomimetic deposition of apatite selected from fluorapatite, hydroxylapatite or mixtures thereof on teeth of vertebrates, comprising

(1) producing at least one partially elastic shaped body, in particular shaped body A, comprising at least one mineralization matrix containing at least one gel containing water-soluble phosphates or phosphates that can be hydrolyzed to form water-soluble phosphates, and producing the shaped body through mixing,

a) for producing at least one mineralization matrix containing the gel, also called phosphate component A, in a first step A, a mixture of

(i) 0.05 to 4 mol/l, 0.5 to 1.5 mol/l water-soluble phosphates or phosphates that can be hydrolyzed to form water-soluble phosphate ions;

(ii) a corresponding amount of water or a mixture of water and an organic solvent;

(iii) optionally at least one 2-hydroxycarboxylic acid having a hydrocarbon residue on C-2, and, optionally, a buffer system, in particular for adjusting the pH value to 2 to 8, preferably 3.5 to 8, more preferably 3.5 to 6, particularly preferably about 5.5 plus/minus 0.5;

(iv) 0 to 6,000 ppm by weight water-soluble fluoride or compound releasing fluorides, in particular 1 to 4,000 ppm by weight, more preferably 500 to 2,500 ppm by weight, particularly preferably about 2,000 ppm by weight plus/minus 500 ppm by weight, and using, in a further step, the mixture produced in a)

b) together with gelatine and optionally glycerol, while heating, to produce the gel,

c) forming of the gel to form the mineralization matrix, optionally solidification.

In both methods, a plane, in particular the envelope, arranged at the outer surface of the at least one mineralization matrix can be formed in a subsequent step d) while the shaped body is being formed.

The pH value of the phosphate solution is between 2.0 and 8.0, preferably between 3.5 and 5,5, and is adjusted using a suitable buffer system. Carboxylic acids, such as lactic acid, but all other buffer systems as well, are particularly preferred for this purpose. The concentration of the buffer is between 0.25 and 4.0 mol/l, preferably between 0.5 and 1.5 mol/l.

The solution is used to produce a gelatine-glycerol gel. The amount of gelatine preferably is 25 to 40% by weight and the amount of glycerol is 5 to 20% by weight with respect to the total composition of aqueous gel. In order to mix the components homogeneously, the preparation is heated to 40 to 90° C., preferably to 50 to 70° C. The thickness of phosphate component A in this context is 50 to 3,000 μm, preferably 200 to 2,000 μm, particularly preferably 300 to 1,500 μm.

Also a subject matter of the invention is a method for producing a calcium ions-containing formulation and a formulation obtainable according to said method for deposition of apatite, in particular for biomimetic deposition of apatite, selected from fluorapatite, hydroxylapatite or mixtures thereof on teeth of vertebrates, comprising

(1) producing at least one partially elastic shaped body, in particular shaped body B, comprising at least one mineralization matrix containing at least one gel containing calcium ions or compounds releasing calcium ions, and producing the shaped body through mixing,

a) for producing at least one mineralization matrix containing the gel, also called calcium component B, in a first step, a mixture of

(i) 0.1 to 2 mol/l calcium ions or compounds releasing calcium ions,

(ii) a corresponding amount of water or a mixture of water and an organic solvent,

(iii) optionally at least one 2-hydroxycarboxylic acid containing a hydrocarbon residue on C-2, and, optionally, a buffer system, in particular for adjusting the pH value to 3.5 to 14, preferably 4.0 to 6.0 or 6.0 to 11.0, preferably 4.0, particularly preferably about 4.0 plus/minus 0.5, and using, in a further step, the mixture produced in a)

b) together with gelatine and optionally glycerol, while heating, to produce the gel;

c) forming to form the mineralization matrix, optionally solidification, and a plane, in particular the envelope, arranged at the outer surface of the at least one mineralization matrix in a subsequent step d), while the shaped body is being formed. The plane or envelope can be produced through the aforementioned cross-linking or through application of a coating. A certain degree of porosity is crucial in the production of the plane or envelope in order to enable the deposition of the bio-composites.

For producing the aforementioned formulations, in b), 5 to 50% by weight gelatine with respect to the total composition of the gel and 0 to 30% by weight glycerol with respect to the total composition of the gel are added each independently in a further step, preferably 25 to 40% by weight gelatine and 5 to 20% by weight glycerol are added to produce the formulation containing the mineralization matrix containing water-soluble phosphates or phosphates that can be hydrolyzed to form water-soluble phosphate ions, and 20 to 40% by weight gelatine and 15 to 25% by weight glycerol are added to produce the formulation containing the mineralization matrix containing calcium ions or compounds releasing calcium ions.

The gelatine-containing formulations in step b) are preferably heated to 40 to 90° C. in order to homogeneously mix the components, preferably, the temperature range is 50 to 70° C.

The solution for producing calcium component B contains a water-soluble calcium salt, e.g. calcium chloride or calcium lactate or calcium gluconate or calcium lacto-gluconate. The listing is inclusive, but not exclusive. The concentration is between 0.1 and 2.0 mol/l, preferably between 0.5 and 1.5 mol/l. The pH value between 4.0 and 14.0, preferably between 6.0 and 11.0, is adjusted using a suitable buffer system. Carboxylic acids, such as ascorbic acid, pyruvic acid, tartaric acid, acetic acid, lactic acid or malic acid, but all other buffer systems just as well, are particularly well-suited for this purpose. The concentration of the buffer is between 0.1 and 3.0 mol/l, preferably between 0.25 and 1.0 mol/l. The solution is used to produce a gelatine-glycerol gel. The amount of gelatine preferably is 20 to 40% by weight with respect to the total composition of aqueous gel and the amount of glycerol is 15 to 25% by weight. Since the calcium-gelatine solution is very tacky even after gelling and thus is unpleasant to handle, a poorly soluble calcium salt is added to improve the texture. Calcium sulfate, calcium apatite, calcium carbonate, calcium oxalate are particularly well-suited. The listing is inclusive, but not exclusive. In order to obtain a particularly homogeneous paste, it is advantageous for the particle sizes to be less than 10 μm. It is preferred to use particles with particle sizes of less than 1 μm. The amount of the poorly soluble calcium salt added preferably is 1 to 50 weight %, very preferably 5 to 30%. In order to mix the components homogeneously, the preparation is heated to 40-90° C., preferably to 50 to 70° C. The thickness of calcium component B in this context is 10 to 3,000 μm, preferably 100 to 1,500 μm, particularly preferably 300 to 1,500 μm, even more preferably 500 to 1,500 μm.

In order to produce the shaped bodies of the formulations, the not-yet-solidified gels are formed and then solidified.

The gels for formation of the mineralization matrix can be formed by filling them in moulds, extrusion, forming into flat elements by painting, distribution, streaking out, pressing through appropriately shaped nozzles, followed by a solidification step. Extrusion of the gels into any shape is preferably possible with pasty gels. The solidification usually proceeds as early as during the cooling process.

For this purpose, the cross-linker is present in a cross-linker solution and is contacted to the gel thus formed. Solutions containing 0.005 to 90% by weight cross-linker in solvent with respect to the total composition, in particular in water or water-containing solvent are preferred, whereby 0.005 to 5% by weight are preferred, 0.1 to 4% by weight are particularly preferred, and about 0.1 to 1% by weight are advantageous. It is preferred to use an aqueous glutardialdehyde solution as cross-linker solution. The cross-linker solution is preferably prepared by adding the cross-linker to the phosphate solution. The preferred time of action is 1 to 200 seconds, particularly preferably 10 to 60 seconds (s). Preferably, the treatment takes approx. 20 seconds. The preferred pH value of the cross-linker solution is between 4.0 and 12.0. Final rinsing with phosphate and/or calcium solution is feasible. Also a subject matter of the invention is a cross-linker solution comprising a phosphate solution or a calcium solution and a content of cross-linker.

Also a subject matter of the invention is a kit comprising a partially elastic shaped body A, in particular a formulation of a partially elastic shaped body A, and a partially elastic shaped body B, in particular a formulation of a partially elastic shaped body B, which each, independently, are present in the form of a three-dimensional body, in particular as a flat element or at least partial negative image of a jaw, whereby (a) partially elastic shaped body A comprises (a1)) at least one mineralization matrix comprising at least one gel, (a2) at least one water-soluble phosphate or phosphates that can be hydrolyzed to form water-soluble phosphate ions, and (a3), optionally, at least one 2-hydroxycarboxylic acid having a hydrocarbon residue on C-2 and, optionally, a buffer system, (a4) water-soluble fluorides or a compound releasing fluorides, (a5), optionally, a content of water or of a mixture of water and an organic solvent;

(b) partially elastic shaped body B comprises (b1) at least one mineralization matrix comprising at least one gel, (b2) water-soluble calcium ions or compounds releasing calcium ions; and

(b3), optionally, at least one 2-hydroxycarboxylic acid having a hydrocarbon residue on C-2 and, optionally, a buffer system, (b4), optionally, water or a mixture of water and an organic solvent.

Kit comprising (I) a shaped body in the form of a flat element having at least two mineralization matrices, optionally separated by a membrane, each independently in the form of a flat element containing the gel at a layer thickness of 50 to 6,000 μm, or (II) two separate shaped bodies each independently in the form of a flat element each having at least one mineralization matrix in the form of a flat element containing the gel, whereby each shaped body independently has a layer thickness of 10 to 3,000 μm, whereby the first shaped body comprising water soluble phosphates or phosphates that can be hydrolyzed to form water-soluble phosphates has a layer thickness of 50 to 3,000 μm, and the second shaped body comprising calcium ions or compounds releasing calcium ions has a layer thickness of 10 to 3,000 μm. According to the alternative, i.e. the shaped body being a negative image of the jaw, the shaped body preferably comprises inner layers of the at least one or two mineralization matrix/matrices that adapt to the teeth. Advantageously, the shaped body can just as well be present in the form of at least a partial negative image of the upper and/or lower jaw.

Moreover, the kit preferably comprises a formulation in the form (a) of an aqueous pre-treatment solution, which is synonymous to mouthwash, comprising water, 0.1 to 30% by weight of a calcium salt that dissolves well in water, in particular 5 to 15% by weight with respect to the total composition, preferably calcium lactate, calcium-chloride, calcium gluconate, calcium lacto-gluconate, a hydrate of the salts or a mixture containing two of the salts, optionally a content of a buffer system, optionally masking agent or flavouring agent and comprising a pH value of 5.0 to 12.0, or the formulation in form b) comprises at least one calcium salt that dissolves well in water, preferably comprising calcium lactate, calcium chloride, calcium gluconate, calcium lacto-gluconate, a hydrate of the salts or a mixture containing two of the salts, optionally a content of a buffer system, optionally a content of masking agent or flavouring agent, and common formulation excipients, such as disintegrants, HPMC, etc., in particular in the form of a water-soluble granulated material, water-soluble pellet, tablets, as sachet, powder and/or granulated material in a releasing means such as a sachet or soluble capsules. Also a subject matter of the invention is the use of a single formulation of form b) together with a kit comprising the formulations according to the invention.

The composition of the kit is described in the following. The mouthwash or pre-treatment solution is composed of 0.1% by weight to 30% by weight of a calcium salt that dissolves well in water, preferably 5% by weight to 15% by weight calcium chloride or calcium lactate or calcium gluconate or calcium lacto-gluconate or other sufficiently soluble calcium salts. The solution is adjusted to a pH value between 5.0 and 12.0, preferably 8.0 to 10.0, using a suitable buffer. To improve the taste, flavouring or complexing of bad tasting compounds is feasible as long as this does not have a detrimental effect on the deposition of apatite. Suitable as buffers are all buffers showing good buffering capacity in said pH range, e.g. EDTA, Tris, HEPES or barbital-acetate buffer, but other buffer systems as well, with Tris being preferred.

It is also preferable for the formulations or mouthwashes to comprise at least one buffer system. Particularly preferred salts of the aforementioned acids are the alkali, alkaline earth and/or zinc salts of citric acid, malic acid, tartaric acid and/or lactic acid or Tris. Particularly preferred salts comprise the cations of sodium, potassium, magnesium and/or zinc of the aforementioned carboxylates.

Preferably, the formulations according to the invention can be used for treatment of sensitive teeth, sensitive dental necks, acid-eroded teeth, cracked teeth, surface-abraded teeth, exposed dental necks, bleached teeth, teeth after treatment of carious tooth regions once (once-a-day) or twice (for example, one-day-mineralization) in order to form an apatite layer, which preferably is homogeneous and essentially crystalline, of 2 to more than or equal to 5 μm in thickness on the treated surfaces. As a matter of principle, the formulation can be used more frequently according to need, for example according to defined intervals.

FIGS. 5a and 5b disclose general embodiments of the shaped bodies according to the invention. FIG. 5a shows two shaped bodies 0, whereby the mineralization matrix 2 comprises the calcium component and the mineralization matrix 1 comprises the phosphate component. The envelope (cross-linking, coating) is indicated by 4 and the optional membrane (layer) by 5. FIG. 5a shows two shaped bodies with one mineralization matrix each and FIG. 5b shows a shaped body having two mineralization matrices in an envelope. The mineralization matrices can just as well each contain calcium or phosphate at different concentrations.

### BRIEF DESCRIPTION OF THE DRAWINGS

FIG. 1 shows the typical surface morphology of the coating at the boundary between coated and uncoated samples after one treatment.

FIG. 2 is an SEM image of the boundary between coated enamel and uncoated enamel.

FIGS. 3a and 3b show before and after toothbrush abrasion on tooth discs treated on half a side.

FIG. 4 is a dentine surface showing growth. The pore-rich dentine surface is covered by a growth of a homogenous layer of needle-like crystals.

FIG. 5a shows two shaped bodies having one mineralization matrix each.

FIG. 5b shows one shaped body having two mineralization matrices in an envelope.

FIG. 6 shows three components: 1) a protective splint (application splint); 2) calcium component (first shaped body I); and 3) phosphate component (further shaped body II)

The invention is illustrated in more detail based on the following examples and figures without limiting the invention to the examples given.

### EXAMPLE 1

For component A containing phosphate ions, a solution containing 29.5 g NaH₂PO₄, 33 g Olaflur, and 27.0 g lactic acid was produced. The pH value was adjusted to 5.4 with 5 N sodium hydroxide solution and the solution was topped up to 250 ml with de-ionized water. A total of 24 ml of the solution and 6 g glycerol and 10 g of 300 Bloom pork rind gelatine were processed while heating to form a viscous solution. A small amount of liquid was placed in a template with a wall thickness of 500 μm and exposed to 2 bar of pressure. After solidification, the strips were removed from the template and cut into 1×1 cm squares.

For component B containing calcium ions, a calcium chloride solution containing 29.4 g calcium chloride dihydrate and 6.3 g lactic acid was prepared. The pH value was adjusted to 4.0 with 5 N sodium hydroxide solution. The solution was topped up to 200 ml with de-ionized water. In order to produce the gel, 21.6 g of the solution and 8.24 g glycerol and 8 g calcium sulfate and 13.6 g 300 Bloom gelatine were mixed and heated. The liquid gel was then spread with a squeegee to a thickness of 1 mm or pressed in a template with a wall thickness of 1 mm. After solidification, the strips were cut into 1×1 cm squares. For the pre-treatment solution (mouthwash), a 0.1 mol Tris buffer was added to a 1 molar calcium chloride solution and the pH was adjusted to 9.0.

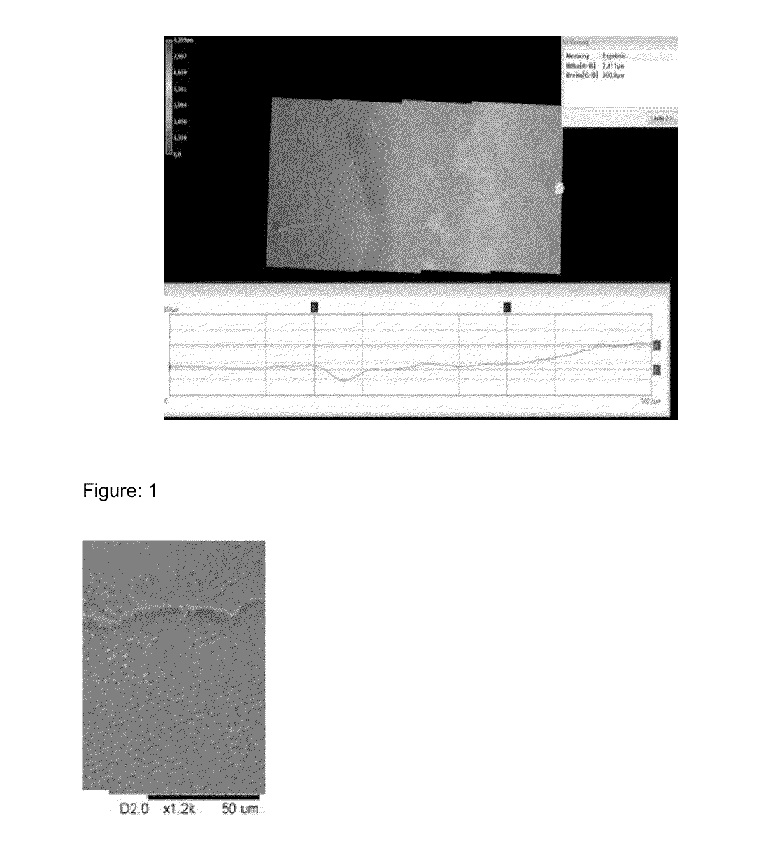

For assessment of the mineralization activity, 6 tooth discs each were etched for 10 s with 1 M HCl, rinsed with the pre-treatment solution, and covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 8 to 16 hours with lukewarm water and a soft toothbrush. After just one treatment, most of the tooth surface is coated by a firmly adhering layer. FIG. 1 shows the typical surface morphology of the coating at the boundary between coated and uncoated sample after one treatment. The layer can be up to 2.5 μm in thickness.

It was evident that the apatite layers grow better when a formulation containing lactic acid, rather than other carboxylic acids, was used.

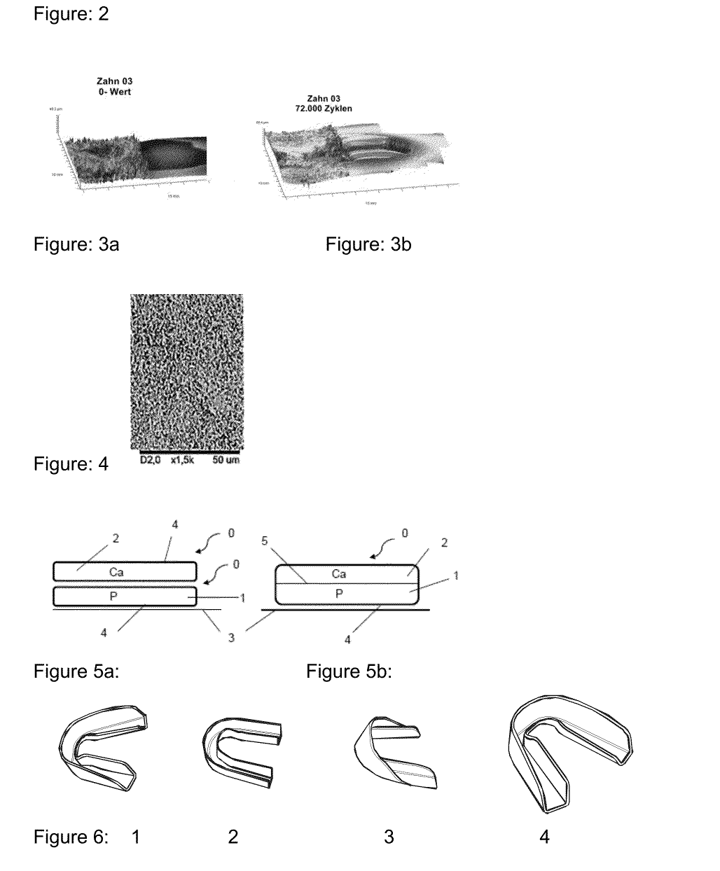

FIG. 1: Typical layers after one application of the mineralization kit 1: Light microscopy image in false colour, 3D microscope made by Keyence, typical layer after one application of the mineralization kit. The colours represent the different heights. The thickness of the layer can be determined from the line scan. Left: untreated dentine (blue) right: treated dentine (green-red). The channel structure disappears under a dense layer (3D microscope, Keyence), which can be up to 2.5 μm in thickness. FIG. 2: SEM image of the boundary between coated enamel and uncoated enamel.

### EXAMPLE 2

The components were produced as described in example 1. However, aside from (example 2i) lactic acid, (example 2a) malic acid, (example 2b) citric acid, (example 2c) tartaric acid, (example 3d) ascorbic acid, (example 2e) trimellitic acid, (example 2f) glycolic acid, (example 2g) salicylic acid, and (example 2h) tropic acid were used as acid component of the buffer system. Provided the acids were sufficiently soluble, the calcium solution and the phosphate solution were adjusted to pH 4.0 and pH 5.2, respectively.

After the application of the gels, a staining test was used to check if a dentine-sealing effect occurred. Acids that were insoluble at the requisite conditions were not tested further.

Principle of the staining test: The samples are measured by colorimetry on the coated and uncoated side after staining with 1% rhodamine solution using a Spectraflash 600 Plus two-channel spectrometer with the CIE L*a*b* system and aperture 3 mm X4SAV. The difference in colour is reported as the delta value (colour difference).

Tris buffer (pH value: 7.2 to 9.0) was used in the Ca gel. A sodium acetate buffer (pH value: 3.7 to 5.7) was used in the P gel.

The results are summarized in Table 1. Accordingly, aside from the calcium salts of lactic acid, the calcium salts of the acids, malic acid, aspartic acid, ascorbic acid, and glycolic acid, are sufficiently soluble and show deposition after application of the kit. Ascorbic acid leads to intensive yellowish discolouration of the teeth though and is therefore not suitable for use, whereas the other acids afforded inferior results as compared to lactic acid. In general, gels coloured yellow are undesirable considering the lack of acceptance by the user. For this reason, glycolic acid, aspartic acid, and malic acid, used as the sole acid, are not preferred in the application. They can be used in combination with lactic acid or lactic acid derivatives though.

TABLE 1
2-Hydroxycarboxylic acids of examples 2a to 2i.

| Carboxylic | Pre-treatment | | | Functional | |
|---|---|---|---|---|---|
| acids | solution | Ca gel | P gel | test (delta E) | pKs |
| Malic acid | pH 8.5 | 0.034 mol = 4.6 g | 0.06 mol = 8.04 g | 42.41 ± 5.39 | 3.46/5.10 |
| | precipitation | precipitation | yellowish-turbid at | | |
| | | | pH 5.25 | | |
| | pH 9.02 clear | 0.017 mol = 2.3 g | 0.03 mol = 4.02 g | | |
| | solution | clear solution, | clear yellow | | |
| | | precipitation after | solution, formation | | |
| | | 24 h | of flakes after 24 h | | |
| | pH 9.53 clear | | | | |
| | solution | | | | |
| Citric acid | pH 9.00 clear | 0.035 mol = 6.67 g | 0.06 mol = 11.52 g | | 3.13/4.76/6.40 |
| | solution | precipitation | yellowish-turbid at | | |
| | | | pH 5.21 | | |
| Tartaric acid | not produced | not produced | Addition of tartaric | | 2.98/4.34 |
| | | | acid leads to | | |
| | | | immediate | | |
| | | | precipitation | | |
| Ascorbic acid | pH 9.00 clear | 0.034 mol = 5.99 g | 0.06 mol = 10.57 g | 56.64 ± 9.85 | 4.37 ± 0.53 |
| | solution, colour | clear, colour | clear, colour | tooth shows | |
| | changes to | changes to | changes | yellow | |
| | yellow | yellow | progressively to | discolouration | |
| | | | yellow | | |
| Aspartic acid | insoluble | 0.034 mol = 4.99 g | 0.06 mol = 7.99 g | 45.96 ± 2.63 | 1.99/3.99/“9.9 0” |
| | | clear (after | turbid, slightly | | |
| | | addition of | yellowish, clear | | |
| | | NaOH) | after addition of | | |
| | | | NaOH | | |
| Trimellitic acid | not produced | 0.03 mol = 3 g | 0.06 mol = 6.6 g | 42.05 ± 11.24 | — |
| | | massive | soluble when | combined with | |
| | | precipitation | warm from pH 4.2 | acetic acid-Ca | |
| | | | | gel | |
| Glycolic acid | not produced | 0.034 mol = 3.16 g | 0.06 mol = 6.37 g | 35.32 ± 4.19 | 3.83 |
| | | gel-like | clear, yellow | combined with | |
| | | precipitation after | solution | acetic acid-Ca | |
| | | addition of NaOH | | gel | |
| Salicylic acid | not produced | insoluble | | | 2.75/12.38 |
| Tropic acid | insoluble | | | | |
| Lactic acid | pH 9.00 clear | 0.034 mol = 3.15 g | 0.06 mol = 5.40 g | 49.83 ± 4.02 | 3.90 |
| | solution | clear | clear | | |

In additional application tests, which also included formulations produced with acetic acid, it was evident that the lactic acid formulations were tolerated better. This has been related, to some extent, to the odour developed by acetic acid or other odorous acids such that the formulations thus produced were perceived as less pleasant.

The tolerability of the carboxylic acids is essential to the application, since the formulations are to remain in the mouth for several hours and should not lead to any skin or mucosal irritations. Another advantage of having lactic acid in the formulations according to the invention is its preservative effect. In order for weak acids to have a preservative effect, the pH of the formulation must be acidic, since only non-dissociated molecules can penetrate through the cell membrane into the inside of microorganisms.

It is therefore particularly preferred according to the invention to use the lactic acid in the phosphate component and preferably in the calcium component.

### EXAMPLE 3

The components were produced as described in example 1.

For assessment of the mineralization activity, 6 tooth discs each were rinsed with the pre-treatment solution and covered with one piece of phosphate gel and one piece of calcium gel each. In order to make the morphological change of the tooth surface more obvious, one half of a disc was taped over first such that only half of the disc can remineralize. The samples were kept in an air-conditioned cabinet at 37° C. and 95% humidity. Grew daily and was subjected to another gel treatment. After three treatments, the tooth surface was basically completely covered by a firmly adhering layer.

The enamel-like stability can be shown by means of toothbrush abrasion tests. After 72,000 homogeneous brush strokes (150 g load) on a tooth sample treated on half of a side, it was evident that the unprotected dentine was abraded markedly more strongly than the unprotected side, which was barely abraded, much like natural enamel. FIGS. 3a and 3b: 3× tooth discs treated on half of a side before (left) and after toothbrush abrasion. It is clearly evident that treatment with the mineralization kit protects from abrasion, since the untreated dentine is abraded strongly—as is evident from a 3D image of biomimetically treated teeth on which fluorapatite was deposited versus untreated teeth, whose tooth surfaces were subsequently abraded by means of a toothbrush. The untreated teeth were abraded strongly.

EXAMPLE 4

For production of the p solution of the P component, 5.9 g Na₂HPO₄, 9.1 g lactic acid, 6.6 g Olaflur, and 0.6 g 5 M NaOH were topped up to 50 ml with de-ionized water to produce. The gel was produced as described in example 1. The calcium solution for the Ca component was produced by dissolving 14.7 g CaCl₂, 3.15 g lactic acid, 10 g 5 M sodium hydroxyde solution in de-ionized water to produce a total of 100 ml of the solution. The gel was produced as described in example 1. The same applies to the pre-treatment solution.

However, the cross-linking proceeded for 2×20 s from both sides with both gels. In this context, the GDA concentration of the Ca cross-linker solution was 0.5% by weight. The P gel was treated for 30 s with a 0.375% GDA solution, which has been produced by mixing the P solution with the appropriate amount of GDA. The gel strips were then only dabbed to dry them.

For assessment of the mineralization activity, 6 tooth discs each were etched for 10 s with 1 M HCl, rinsed with the pre-treatment solution, and then covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 8 to 12 hours with lukewarm water and a soft toothbrush. After just one treatment, most of the tooth surface is coated by a firmly adhering layer.

A largely homogeneous layer of small needle-like crystals was seen in the electron microscope.

FIG. 4: Dentine surface showing growth. The pore-rich dentine surface is covered by a growth of a homogeneous layer of needle-like crystals. After treatment with the mineralization kit (GDA cross-linking on both sides).

FIG. 5a, 5b: FIG. 5a shows two shaped bodies having one mineralization matrix each and FIG. 5b shows one shaped body having two mineralization matrices in an envelope.

FIG. 6: Shows three components: 1. Protective splint (application splint), 2. Calcium component (first shaped body I), 3. Phosphate component (further shaped body II). 1 and 2 can be combined into one mould (application splint and the first shaped body form a unit), 3 (further shaped body) must not be added until shortly before the start of mineralization.

1. A formulation suitable for depositing apatite selected from fluorapatite, hydroxyapatite and mixtures thereof on vertebrate teeth comprising
at least one partially elastic shaped body comprising at least one mineralization matrix containing at least one gel, whereby the gel comprises at least one carboxylic acid or a mixture of carboxylic acids selected from 2-hydroxycarboxylic acids substituted on C-2 by a hydrocarbon-residue, and, optionally, a buffer system, and
a) the at least one mineralization matrix containing a gel comprises water-soluble phosphates or hydrolysable phosphates that form water-soluble phosphate ions and has a pH value of 2 to 8, and
b) the at least one or a second mineralization matrix comprises a second gel comprising calcium ions or compounds releasing calcium ions and has a pH value of 3.5 to 14.

2. The formulation according to claim 1 wherein the shaped body comprises a reduced solubility in aqueous media as compared to the mineralization matrix in at least one plane, at least partly, whereby the plane serves as a membrane.

3. The formulation according to claim 1 wherein the at least one mineralization matrix is present in a first shaped body I and the second mineralization matrix is present in a second shaped body II.

4. The formulation according to claim 1 wherein
in a), the at least one mineralization matrix comprises a gel comprising
(i) water-soluble phosphates or hydrolysable phosphates that form water-soluble phosphate ions,
(ii) a content of water or of a mixture of water and an organic solvent,
(iii) at least one carboxylic acid or a mixture of carboxylic acids, in particular comprising lactic acid, and optionally a buffer system, and/or the second mineralization matrix or the at least one mineralization matrix, in (b), comprises a second gel comprising
(i) calcium ions or compounds releasing calcium ions,
(ii) optionally, water or a mixture of water and an organic solvent, and
(iii) at least one carboxylic acid or a mixture of carboxylic acids and optionally a buffer system.

5. The formulation according to claim 1 wherein the at least one mineralization matrix comprises a gel comprising at least one water-soluble fluoride or one compound releasing fluorides, optionally comprising at least one non-substituted or substituted alkyl groups-comprising quaternary mono- or poly-ammonium compound, such as N,N,N′-tris(2-hydroxyethyl)-N′-octadecyl-1,3-diaminopropandihydrofluoride (Olaflur), aminefluorides, such as Oleaflur, Decaflur, ethanolamine hydrofluoride, or water-soluble inorganic fluorides such as alkali fluorides, sodium fluoride, potassium fluoride, tin fluoride, ammonium fluoride or inorganic fluorides releasing fluorides, such as zinc fluoride, zinc hydroxyfluoride.

6. The formulation according to claim 1 wherein the gel comprises at least one gel-forming agent selected from denatured collagen, hydrocolloids, polypeptides, protein hydrolysis products, polysaccharides, polyacrylates or mixtures comprising at least two of the aforementioned gel-forming agents, whereby the gel comprises gelatine and a polyol, the adducts thereof and/or the conversion products thereof.

7. The formulation according to claim 1 wherein a) the at least one mineralization matrix comprising at least one gel is present in at least one partially elastic shaped body in the form of a flat element or at least partial negative image of a jaw, whereby the shaped body comprises, in particular, at least two planes that are arranged on the outer surface or comprises at least one partial outer envelope that possesses reduced solubility with respect to aqueous media as compared to the mineralization matrix, and/or
b) the at least one or two mineralization matrices comprising at least one gel is/are present in at least one partially elastic shaped body in the form of a flat element or at least partial negative image of a jaw.

8. The formulation according to claim 1 wherein the planes or envelope form the outer boundary of the mineralization matrix.

9. The formulation according to claim 1 comprising two separate shaped bodies, each independently in the form of a flat element each having at least one mineralization matrix containing a gel, whereby each shaped body independently has a layer thickness of 10 to 3,000 μm, whereby the first shaped body comprising water soluble phosphates or hydrolysable phosphates that form water-soluble phosphate ions has a layer thickness of 50 to 3,000 μm, and/or the second shaped body comprising calcium ions or compounds releasing calcium ions has a layer thickness of 10 to 3,000 μm.

10. A method for producing a formulation according to claim 1, suitable for biomimetic deposition of apatite selected from fluorapatite, hydroxylapatite or mixtures thereof on vertebrate teeth comprising
(1) producing at least one partially elastic shaped body comprising at least one mineralization matrix containing at least one gel containing water-soluble phosphates or hydrolysable phosphates that form water-soluble phosphates, and producing the shaped body through mixing,
a) for producing at least one mineralization matrix containing the gel, in a first step, a mixture of
(i) 0.05 to 4 mol/l water-soluble phosphates or hydrolysable phosphates that form water-soluble phosphate ions;
(ii) a corresponding amount of water or of a mixture of water and an organic solvent;
(iii) at least one 2-hydroxycarboxylic acid substituted by a hydrocarbon on C-2 and, optionally, a buffer system,
(iv) 0 to 6,000 ppm by weight water-soluble fluoride or compound releasing fluorides, and using, in a further step, the mixture produced in a)
b) together with gelatine and optionally glycerol, while heating, to produce the gel,
c) forming the gel to form the mineralization matrix, optionally solidification.

11. The method for producing a formulation according to claim 1, suitable for biomimetic deposition of apatite selected from fluorapatite, hydroxylapatite or mixtures thereof on vertebrate teeth comprising
(1) producing at least one partially elastic shaped body comprising at least one mineralization matrix containing at least one gel containing calcium ions or compounds releasing calcium ions, and producing the shaped body through mixing,
a) for producing at least one mineralization matrix containing the gel, in a first step, a mixture of
(i) 0.1 to 2 mol/l calcium ions or compounds releasing calcium ions;
(ii) a corresponding amount of water or of a mixture of water and an organic solvent;
(iii) at least one 2-hydroxycarboxylic acid substituted by a hydrocarbon on C-2 and, optionally, a buffer system, and using, in a further step, the mixture produced in a)
b) together with gelatine and optionally glycerol, while heating, to produce the gel,
c) forming the gel to form the mineralization matrix, optionally solidification.

12. The method according to claim 10 wherein the shaped body in (1) possesses, at least in part in at least one plane, reduced solubility with respect to aqueous media as compared to the mineralization matrix, and in that the shaped body is produced by d) forming a plane of the at least one mineralization matrix that is arranged on an outer surface, while the shaped body is being formed.

13. The method according to claim 10 wherein the gel produced in further step b) is being formed, in particular into a flat element or an individual three-dimensional element, and in that the gel can be solidified in said shape.

14. A shaped body obtainable according to a method according to claim 10.

15. A kit comprising at least one formulation comprising a partially elastic shaped body A and a separate partially elastic shaped body B, each independently comprising a formulation according to claim 1, whereby
(a) partially elastic shaped body A comprises
(a1) at least one mineralization matrix comprising at least one gel,
(a2) at least one water-soluble phosphate or hydrolysable phosphates that form water-soluble phosphate ions, and
(a3) at least one 2-hydroxycarboxylic acid substituted by hydrocarbon on C-2 and, optionally, a buffer system
(a4) optionally, water-soluble fluorides or a compound releasing fluorides
(a5), optionally, a content of water or of a mixture of water and an organic solvent
(b) partially elastic shaped body B comprises
(b1) at least one mineralization matrix comprising at least one gel,
(b2) water-soluble calcium ions or compounds releasing calcium ions, and
(b3) at least one 2-hydroxycarboxylic acid substituted by a hydrocarbon on C-2 and, optionally, a buffer system
(b4), optionally, water or of a mixture of water and an organic solvent, or a partially elastic shaped body C comprising a formulation, whereby the
(c) partially elastic shaped body C comprises
(c1) at least one mineralization matrix comprising at least one gel,
(c1.1) at least one water-soluble phosphate or hydrolysable phosphates that form water-soluble phosphate ions, and
(c1.2) at least one 2-hydroxycarboxylic acid substituted by a hydrocarbon on C-2 and, optionally, a buffer system
(c1.3) optionally, water-soluble fluorides or a compound releasing fluorides
(c1.4), optionally, a content of water or of a mixture of water and an organic solvent
(c2) optionally, a membrane (layer)
(c3) at least one mineralization matrix comprising at least one gel,
(c3.1) water-soluble calcium ions or compounds releasing calcium ions, and
(c3.2) at least one 2-hydroxycarboxylic acid substituted by a hydrocarbon on C-2 and, optionally, a buffer system
(c3.3) optionally, water or a mixture of water and an organic solvent, whereby the layer structure of the partially elastic shaped body C is c1 and c3 or c1, c2, and c3.
